(19) United States
(12) Patent Application Publication
(10) Pub. No.: US 2012/0027852 A1
(43) Pub. Date: Feb. 2, 2012

(54) PHARMACEUTICAL COMPOSITION CONTAINING A "LIMUS" FAMILY IMMUNOSUPPRESSIVE MACROLIDE

(75) Inventors: Frantz Deschamps, Nancy (FR); Catherine Herry, Saint-ouen Du Tilleul (FR); Jennifer Jung, Dieulouard (FR); Fabrice Leboeuf, Saint Max (FR)

(73) Assignee: ETHYPHARM, Saint-Cloud (FR)

(21) Appl. No.: 13/262,599

(22) PCT Filed: Mar. 31, 2010
(86) PCT No.: PCT/EP2010/054277

§ 371 Date: Sep. 30, 2011

(30) Foreign Application Priority Data
Mar. 31, 2009 (FR) 0952049

(51) Int. Cl. A61K 9/48; A61P 37/06; A61K 9/20; C07D 498/18; A61K 9/14; A61K 31/436

(52) U.S. Cl. 424/452; 424/490; 424/278.1; 424/465; 540/456

(57) ABSTRACT

A pharmaceutical formulation includes a Limus family immunosuppressive macrolide on a pharmaceutically acceptable excipient, which may be compounded as suitable for oral administration.

The present invention relates to a novel orally administered pharmaceutical composition essentially containing fine crystalline particles of a macrocyclic lactone belonging to the therapeutic class of immunosuppressors acting on immunophilins, in particular a “limus” family immunosuppressive macrolide and one or more pharmaceutically acceptable excipients.

More particularly, the present invention relates to excipient/“limus” family immunosuppressive macrolide formulations and to tablets containing said excipient/“limus” family immunosuppressive macrolide formulations.

The present invention also relates to a method for preparing excipient/“limus” family immunosuppressive macrolide formulations.

“Limus” family immunosuppressive macrolides are represented by tacrolimus, sirolimus and analogues thereof such as temsirolimus.

Tacrolimus, also known as FK-506 or Fujimycin, is synthesized by a bacterium, Streptomyces tsukubaensis. This molecule is represented by formula (I) and has the chemical name 5,6,8,11,12,13,14,15,16,17,18,19,24,25,26,26a-hexadecahydro-5,19-dihydroxy-3-[2(4-hydroxy-3-methoxycyclohexyl)-1-methylethenyl]-14,16-dimethoxy-4,10,12,18-tetramethyl-8-(2-propenyl)-15,19-epoxy-3H-pyrido[2,1-c][1,4]oxaazacyclotricosine-1,7,20,21(4H,23H)-tetrone.

Tacrolimus sold under the name Prograf® is provided in the form of a solid dispersion obtained by co-precipitation in the presence of organic solvents.

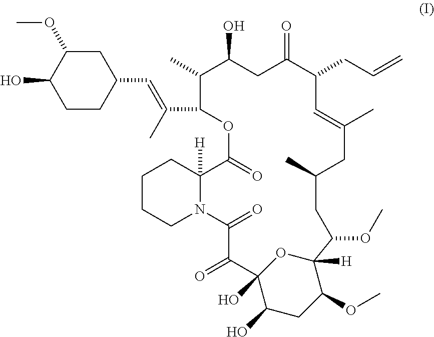

Sirolimus or rapamycin is an immunosuppressive lactam macrolide that is produced by Streptomyces hygroscopicus. U.S. Pat. No. 3,929,992 describes the preparation of sirolimus. This molecule is represented by formula (II) and has the chemical name (3S,6R,7E,9R,10R,12R,14S,15E,17E,19E,21S,23S,26R,27R,34 aS)-9,10,12,13,14,21,22,23,24,25,26,27,32,33,34,34a-hexadecahydro-9,27-dihydroxy-3-[(1R)-2-[(1S,3R,4R)-4-hydroxy-3-methoxycyclohexyl]-1-methylethyl]-10,21-dimethoxy-6,8,12,14,20,26-hexamethyl-23,27-epoxy-3H-pyrido[2,1-c][1,4]-oxaazacyclohentriacontine-1,5,11,28,29(4H,6H,31H)-pentone.

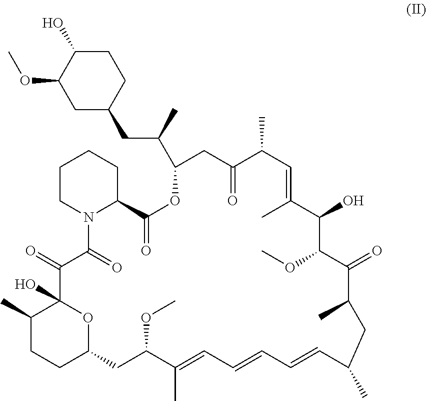

Sirolimus used to prevent rejection of kidney transplants has been marketed under the name Rapamune®, a patent medicine provided in the form of coated tablets (1 mg or 2 mg) in which sirolimus nanodispersed by Nanocrystal® technology in the presence of poloxamer 188 is found in one of the layers of the tablet's coating. Nanocrystal® technology is described as a wet grinding technology that produces a dispersion of nanoparticles in an aqueous medium containing stabilizers. It is thus necessary to convert this dispersion into dry form to produce a stable commercial dosage form, work which can be complex to develop and which can risk degradation or loss of the active ingredient during steps of conversion into a dry form.

Oral administration of “limus” family immunosuppressive macrolides is limited by their very low and variable bioavailability. These compounds are difficult to formulate because they are highly insoluble in biological environments.

Patent application WO2007/091109 describes a composition in granule form containing amorphous tacrolimus dissolved beforehand in ethanol and containing at least one diluting excipient and one binding excipient.

Patent EP0868911 relates to sirolimus compositions containing a core and a sugar coating, wherein said sugar coating contains sirolimus, one or more sugars and one or more surface modifying agents.

Patent EP0839028 relates to compositions of amorphous rapamycin in the form of a solid dispersion in a water-soluble polymer or a cyclodextrin.

Patent application EP1938800 relates to sirolimus nanodispersions containing a surface modifier and sirolimus particles of an average size between 400 nm and 1200 nm.

In general, the nanonization of an active ingredient that is relatively insoluble in water improves its apparent solubility. However, several disadvantages result from this decrease in size. Nanonized particles are not stable and tend to agglomerate. Thus, they are most often produced in the form of dispersions in an aqueous medium containing stabilizers. In addition, grinding to sub-micron dimensions by mechanical processes has a high risk of changing the crystallinity and/or polymorphism of the active ingredient with a risk of producing a large proportion of the amorphous form. However, the amorphous form is less stable than the crystalline form and will evolve toward a crystalline form over time with a potential impact on behavior in dissolution and in man.

Patent application WO2007/067566 describes the preparation of a sirolimus derivative in crystalline form from raw material such as that obtained according to the method described in U.S. Pat. No. 5,985,325. The size of the sirolimus derivative particles is greater than 30 μm.

This patent application describes a standard crystallization method in which the macrolide is purified after dissolution in an ethyl acetate solution heated to 52-58° C., a solution which is then filtered. A precipitation solvent is then added.

Furthermore, it is specified in the text of this application that the crystallinity of sirolimus or of its derivatives contributes to the general quality of the compound, in particular in terms of stability to oxidative degradation; amorphous or partially crystalline forms are subject to rapid oxidative degradation.

It is thus important to be able to prepare a crystalline form of sirolimus, in particular by means of a simplified method that does not involve organic solvents and that produces fine particles, preferably sub-micron particles.

Thus, one object of the present invention relates to an excipient/compound formulation characterized in that it contains particles of at least one pharmaceutically acceptable excipient on which fine crystalline particles of a “limus” family immunosuppressive macrolide according to the invention are captured.

Another object of the invention relates to an orally administered pharmaceutical composition containing a “limus” family immunosuppressive macrolide comprising:
(a) particles of at least one pharmaceutically acceptable excipient,
(b) on which fine crystalline particles of a “limus” family immunosuppressive macrolide according to the invention are captured, wherein said pharmaceutical composition is in the form of a gelatin capsule.

Another object of the invention relates to an orally administered pharmaceutical composition containing a “limus” family immunosuppressive macrolide in the form of a gelatin capsule or a tablet.

Another object of the invention relates to a method for preparing an excipient/compound of the invention formulation.

Another object of the invention relates to a method for preparing an orally administered pharmaceutical composition according to the invention.

Definitions in the Context of the Present Invention:

The expression “fine particles” of a “limus” family immunosuppressive macrolide refers to particles of an average size smaller than a few microns, preferably of sub-micron size. The fine particles preferably have a volume mean diameter (dᵥ(0.5)) smaller than 2 μm, more preferably smaller than 1 μm, particularly preferably smaller than 500 nm. Ninety percent of the fine particles preferably have a volume diameter (dᵥ(0.9)) smaller than 5 μm, more preferably smaller than 2 μm.

In the context of the present invention, the terms “crystalline form” and “crystalline particle” refer to a compound in stable polymorphic form.

“Polymorphic form” refers to an organized structure involving only molecules of the compound and having a characteristic crystalline form.

According to the present invention, “free of solvent” means not prepared in dispersion form, in for example water, ethanol, isopropanol or mixtures thereof.

According to the present invention, “free of surface modifying agents” means free of agents used to disperse the compound in a solvent and to increase the wettability of the compound, such as for example gelatin, casein, lecithin, acacia gum, stearic acid, calcium stearate, carboxymethyl cellulose and derivatives thereof, methyl cellulose, hydroxyethyl cellulose, hydroxypropyl cellulose or hydroxypropyl methylcellulose phthalate.

According to the present invention, “compound” refers to a “limus” family immunosuppressive macrolide.

According to the present invention, “excipient/compound formulation” refers to fine crystalline particles of a “limus” family immunosuppressive macrolide captured on particles of at least one pharmaceutically acceptable excipient. The excipient particles can be directly compressible powders or granules of a granule size between 75 μm and 600 μm.

According to the present invention, “captured” means deposited uniformly on the surface and/or trapped within the pores of the excipient particles.

“Fluid at supercritical pressure” means either a supercritical fluid, i.e., a fluid carried at a pressure and temperature greater than its critical pressure and temperature, respectively, in the case of a pure substance, or to a representative point (pressure, temperature) located beyond the range of critical points represented on a diagram (pressure, temperature) in the case of a mixture; or a so-called “subcritical” fluid, i.e., a fluid in a state characterized either by a pressure greater than the critical pressure and by a temperature lower than the critical temperature in the case of a pure substance, or by a pressure greater than the critical pressures and a temperature lower than the critical temperatures of the components in the case of a mixture. It should be noted that the physicochemical properties of carbon dioxide as well as its critical parameters (critical pressure: 7.4 MPa and critical temperature: 31° C.) make it the preferred fluid in many applications, in addition to the fact that it is nontoxic and it is available at very low cost in very large quantities.

### DESCRIPTION OF THE FIGURES

FIG. 1.1 is a diagram of the general operation of the fine crystalline particle production system according to the invention.

FIG. 1.2 is a diagram showing the results of differential scanning calorimetry (DSC) for characterizing the solid state of sirolimus.

FIG. 2.1 is a thermogram of sirolimus/mannitol formulations (test 2-1) and the corresponding physical mixture.

FIG. 2-2 is a thermogram of sirolimus/lactose formulations (test 2-2) and the corresponding physical mixture.

FIG. 2-3 is a thermogram of microcrystalline sirolimus/cellulose formulations (test 2-3) and the corresponding physical mixture.

FIG. 2-4 is a diagram comparing observations with the scanning electron microscope for pure mannitol (top) and the sirolimus/mannitol formulation (test 2-1) (bottom).

FIG. 2-5 is a diagram comparing observations with the scanning electron microscope for pure lactose (top) and sirolimus formulation/lactose (test 2-2) (bottom).

FIG. 2-6 is a diagram comparing observations with the scanning electron microscope for pure microcrystalline cellulose (top) and the microcrystalline sirolimus/cellulose formulation (test 2-3) (bottom).

FIG. 2-7 is a diagram showing the particle size distribution for the sirolimus/mannitol formulation (test 2-1).

FIG. 2-8 is a diagram showing the particle size distribution for the sirolimus/lactose formulation (test 2-2).

FIG. 2-9 is a diagram comparing in vitro dissolution curves of sirolimus/excipient formulations packaged in gelatin capsules compared to 2 mg Rapamune® tablets.

FIG. 3-1 is a diagram comparing in vitro dissolution curves of sirolimus/excipient formulations packaged in tablet form compared to 2 mg Rapamune® tablets.

### DETAILED DESCRIPTION OF THE INVENTION

A first object of the invention relates to an excipient/compound formulation characterized in that it contains particles of at least one pharmaceutically acceptable excipient on which fine crystalline particles of a “limus” family immunosuppressive macrolide according to the invention are captured.

The particles of pharmaceutically acceptable excipients can be of any chemical nature and are preferentially made of a sugar, a polyol, starch, a cellulose derivative, magnesium aluminosilicate or mixtures thereof.

The excipient particles have a granule size between 75 microns and 600 microns, preferably between 300 microns and 600 microns.

The pharmaceutically acceptable excipients are advantageously selected from lactose (Pharmatose DCL21), microcrystalline cellulose (Avicel PH 200) and mannitol (Pearlitol 200 SD).

The load of the “limus” family immunosuppressive macrolide on the pharmaceutically acceptable excipient particles is between 0.5% and 10%, preferentially between 1% and 4%.

A second object of the invention relates to an orally administered pharmaceutical composition containing a “limus” family immunosuppressive macrolide comprising:
(a) particles of at least one pharmaceutically acceptable excipient,
(b) on which fine crystalline particles of a “limus” family immunosuppressive macrolide according to the invention are captured.

A pharmaceutical composition according to the invention can be provided in gelatin capsule form or tablet form.

According to a first embodiment of the invention, a pharmaceutical composition according to the invention consists of the excipient/compound formulation according to the invention packaged in gelatin capsule form.

According to a second embodiment of the invention, a pharmaceutical composition according to the invention is a tablet containing only the excipient/compound formulation according to the invention. Advantageously, the pharmaceutically acceptable excipients used to prepare the excipient/compound formulation according to the invention are then preferentially selected from the excipients known to those persons skilled in the art to possess satisfactory compression properties.

The tablets of the invention can optionally include one or more other excipients selected from the group comprised of diluents, binders, antistatic agents, lubricants, permeabilizing agents, pH modifiers, antioxidants, sweeteners, flavorings and mixtures thereof. Advantageously, a tablet of the invention consists of the excipient/compound formulation according to the invention and one or more excipients selected from the group comprised of diluents, binders, antistatic agents, lubricants, permeabilizing agents, pH modifiers, antioxidants, sweeteners, flavorings and mixtures thereof.

The proportion of other excipients compared to the excipient/compound formulation of the invention in the tablet of the invention can be variable.

Advantageously, the mixture of excipients comprises:
one or more diluents guaranteeing a tablet mass that matches the manufacturing constraints. All pharmaceutically acceptable excipients described as diluents can be used including cellulose derivatives and lactose derivatives,
one or more binders for producing tablets of suitable physical properties (hardness, friability). All pharmaceutically acceptable excipients described as binders can be used including celluloses, calcium phosphates and lactose,
antistatic agents, lubricants or glidants can be added to improve the properties of the tablet mixture, including silica derivatives, talc, magnesium stearate and sodium stearyl fumarate,
pH modifiers for creating a micro-pH around the molecule, including basic agents such as sodium bicarbonate or calcium carbonate, or acids such as citric acid or fumaric acid,
colorants, sweeteners and flavorings can also be added according to the needs of the formulation,
antioxidants.

A pharmaceutical composition of the invention in tablet form preferably has a rate of dissolution greater than 60%, in a particularly preferred way greater than 70%, in 10 min.

A pharmaceutical composition of the invention in tablet form preferably has a rate of dissolution greater than 80%, in a particularly preferred way greater than 90%, in 20 min.

A pharmaceutical composition of the invention in tablet form preferably has a rate of dissolution greater than 90%, in a particularly preferred way greater than 95%, in 30 min.

These rates of dissolution apply preferably in a dissolution medium comprised of 0.4% sodium dodecyl sulfate (SDS) in water in accordance with the European Pharmacopeia.

A pharmaceutical composition of the invention advantageously comprises 1 mg or 2 mg of compound.

The present invention also relates to a method for preparing the fine crystalline particles of the compound of the invention and the excipient/compound formulations of the invention.

The fine Crystalline particles of the compound of the invention and the excipient/compound formulations of the invention are obtained under particularly advantageous conditions by means of a method using a fluid at supercritical pressure. Among the methods for producing fine particles using a supercritical fluid, the method known as rapid expansion of supercritical solutions (RESS) described in the documents U.S. Pat. No. 4,582,731 and WO01/43853 can be cited, for example.

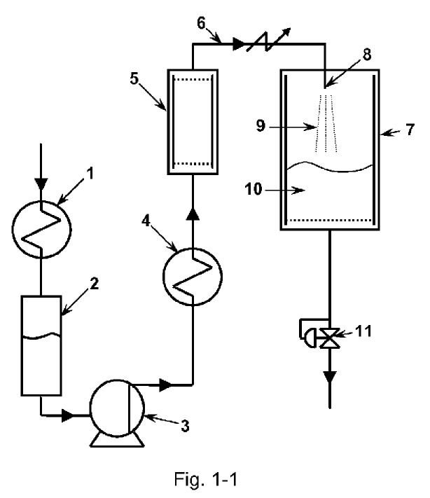

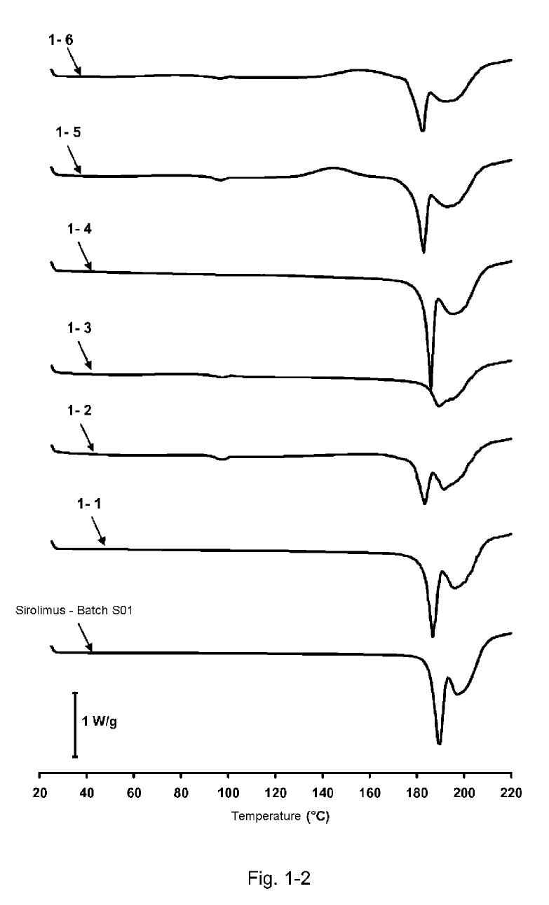

The general operation of the system according to FIG. 1-1 will be described first, followed by a description of the specific operation of this system for preparing the crystalline form of a “limus” compound, as well as the means implemented to obtain an excipient/sirolimus formulation comprised of particles of excipients on which crystalline particles of a “limus” compound are captured.

According to the most common implementation of the RESS technique, the compound to be micronized or nanonized is placed in an extraction cell (tube closed at the ends by stainless steel frits to avoid entrainment of the compound by the fluid). This cell is placed inside an extraction autoclave (5) heated at the selected extraction temperature. The fluid is pumped (3) from the storage vessel (2) and crosses a hot exchanger (4) set at the desired extraction temperature before entering the extraction autoclave. The solution of compound solubilized in the fluid at supercritical pressure leaving the extraction autoclave is sent toward a spraying enclosure (7) in which the solution is suddenly depressurized at a given temperature and pressure through a nozzle (8). A heating device (6) located immediately upstream of the spraying autoclave regulates the pre-expansion temperature of the fluid. The drop in pressure through the nozzle induces a sudden drop in the solubility of the compound in the fluid, leading to the formation of fine particles within the spraying enclosure. This spraying enclosure is generally operated at atmospheric pressure.

In an advantageous implementation, the spraying enclosure contains a collection basket whose bottom is closed by a filter in order to avoid the entrainment of the fine particles formed by the fluid. The fluid then is discharged into the atmosphere or optionally recompressed and recycled. The RESS method thus makes it possible to produce in one step, in the absence of solvent and/or of surfactants, dry fine particles of a compound.

In the context of the present invention, it became evident in a surprising manner that the use of a pressure appreciably greater than atmospheric pressure in the spraying enclosure and set using the upstream pressure regulation valve (11) makes it possible, independently of the pre-expansion temperature set by the heating device (6), to obtain fine crystalline particles of a “limus” family immunosuppressive macrolide in a solid state near to that of the starting crystalline compound, with no or with little amorphous phase, which should ensure the long-term stability of the solid state of the “limus” family immunosuppressive macrolide within the particles since the active ingredient in a stable crystalline form is obtained without risk of recrystallization of an amorphous phase, as well as ensure its chemical stability insofar as the presence of the amorphous phase in “limus” family immunosuppressive macrolide powders is known as a factor that decreases the stability of this type of active ingredient.

Another object of the invention thus relates to a method for preparing fine crystalline particles of a “limus” family immunosuppressive macrolide containing the following steps:
solubilization of a “limus” family immunosuppressive macrolide in a fluid at supercritical pressure in an extraction autoclave (5),
sudden depressurization of the solution obtained in the preceding step through a nozzle (8) in a spraying enclosure (7), wherein the pressure in the spraying enclosure is appreciably greater than atmospheric pressure.

Preferably, the fluid used is CO₂, carried at a pressure greater than its critical pressure (7.4 MPa), between 10 MPa and 100 MPa, and preferably between 10 MPa and 40 MPa, and at a temperature greater than its critical temperature (31° C.), preferably between 35° C. and 80° C., in the extraction autoclave.

Preferably, precipitation of the “limus” family immunosuppressive macrolide is obtained by expansion of the solution of said macrolide in supercritical CO₂ carried at a pre-expansion temperature of 60° C. to 140° C., and preferably between 60° C. and 100° C.

Preferably, the pressure in the spraying enclosure (7) is between 1 MPa and 7 MPa, more preferably between 1 MPa and 4 MPa.

Another object of the invention relates to a method for preparing the excipient/compound of the invention formulation.

The RESS technique, in particular the method for preparing fine solid particles of the invention described above, is then implemented with a step of capturing the particles formed on a bed of excipient (10) placed in the spraying enclosure (7). This particular implementation, described in patent EP1239938, makes it possible to quite effectively capture the particles formed during the process. Moreover, this implementation quite significantly improves the usability of the fine particles of “limus” family immunosuppressive macrolide compared to the standard implementation of the RESS technique, wherein the fine particles are deposited on particles of at least one pharmaceutically acceptable excipient as described above.

In a preferred implementation of the method for preparing the excipient/compound of the invention formulation:
the fluid used is CO₂, carried at a pressure greater than its critical pressure (7.4 MPa), between 10 MPa and 100 MPa, and preferably between 10 MPa and 40 MPa, and at a temperature greater than its critical temperature (31° C.), preferably between 35° C. and 80° C., in the extraction autoclave,
the precipitation of the “limus” family immunosuppressive macrolide is achieved by expansion of the solution of said macrolide in supercritical CO₂ carried at a pre-expansion temperature of 60° C. to 140° C., and preferably between 60° C. and 100° C.,
the pressure in the spraying enclosure (7) is between 1 MPa and 7 MPa, and preferably between 1 MPa and 4 MPa,
the fine crystalline particles of the “limus” family immunosuppressive macrolide formed are captured on a bed of at least one pharmaceutically acceptable excipient (10) placed in the spraying enclosure (7).

Another object of the invention relates to a pharmaceutical composition of the invention for the use of same as a preventive and/or curative immunosuppressive treatment, in particular to treat and/or to prevent rejection after an organ transplant, for example a kidney transplant.

Another object of the invention relates to a method of treatment comprising the administration of a pharmaceutical composition of the invention to a patient in need of such treatment, preferably a patient who has received an organ transplant, for example a kidney transplant, in a quantity sufficient to treat and/or to prevent rejection of the transplanted organ.

The present invention is illustrated in greater detail in the descriptive and nonrestrictive examples below.

### EXAMPLES

### Example 1

### Production of Pure Sirolimus Particles by the RESS method

The RESS method is implemented for the production of pure sirolimus particles, with no excipient in the spraying enclosure, with laboratory-scale equipment corresponding to the diagram described in FIG. 1-1.

0.5 g of sirolimus (Batch S01) mixed with approximately 6 g of 1 mm-diameter glass beads is placed in a 10 ml extraction cell. Said cell is placed inside the extraction autoclave (5) heated at a temperature of 60° C. After initiation of the system, carbon dioxide in a supercritical state, at a pressure of 40 MPa, a temperature of 60° C. and a flow rate of 0.3 kg/h, percolates through the extraction cell to extract the sirolimus. The solution of sirolimus in supercritical carbon dioxide is released through a spray nozzle (8) comprised of PEEK capillary tubing with an internal diameter of 63 μm and a length suited to the pressure and flow rate of the system. The pre-expansion temperature is regulated via a heating device (6) located immediately upstream of the nozzle. The spraying enclosure (7), with a useful interior volume of 65 ml, is heated at 60° C. The pressure within the spraying enclosure is regulated by an upstream pressure regulation valve (11). After running for 5 hours, the CO₂ pump is stopped and the pressure in the spraying enclosure is gradually decreased to atmospheric pressure over approximately 30 minutes before the compound is collected.

Two series of three tests were carried out. Each series corresponds to a pre-expansion temperature of the supercritical solution.

More precisely, the first series of tests is conducted with a pre-expansion temperature of 60° C. The release through the nozzle is very brief and is generally assumed isenthalpic. Consequently, those persons skilled in the art can determine that a release under these conditions leads to the presence of a mixture of liquid CO₂ and CO₂ gas immediately downstream from the nozzle. The second series of tests is conducted with a pre-expansion temperature of 140° C., which is approximately the minimal pre-expansion temperature for the CO₂ to be completely in a gas state downstream from the spray nozzle.

For each series, tests are conducted with pressures in the spraying enclosure of 3 MPa, 1 MPa and 0.1 MPa. Table 1-1 summarizes the parameters used for the six tests.

TABLE 1-1
Conditions for tests 1-1 to 1-6

| | Pre-expansion temperature | Pressure in |
|---|---|---|
| | of the supercritical | the spraying |
| Test number | solution (° C.) | enclosure (MPa) |
| 1-1 | 60 | 3 |
| 1-2 | 60 | 1 |
| 1-3 | 60 | 0.1 |
| 1-4 | 140 | 3 |
| 1-5 | 140 | 1 |
| 1-6 | 140 | 0.1 |

For each test, a mass of approximately 150 mg of sirolimus was extracted and a sample of approximately 100 mg of sirolimus particles was collected in the spraying enclosure.

Results

The fine sirolimus particles obtained by the RESS method were analyzed by differential scanning calorimetry (DSC) to characterize the solid state of the sirolimus within the particles and by high-performance liquid chromatography (HPLC) to quantify the titer and profile of the relative substances.

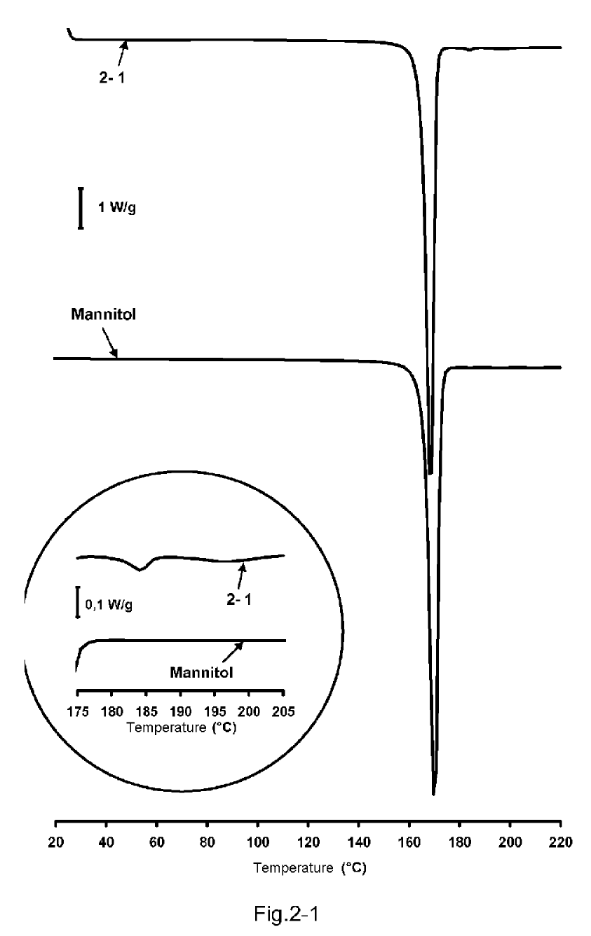

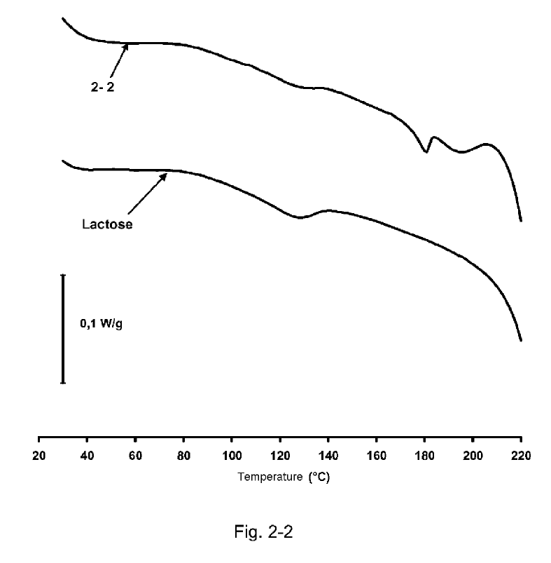

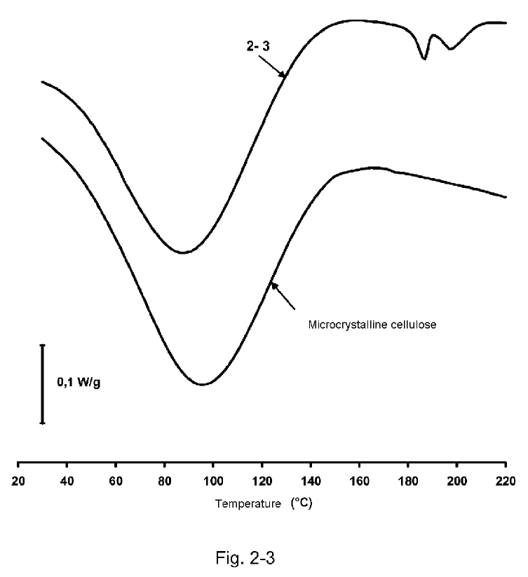

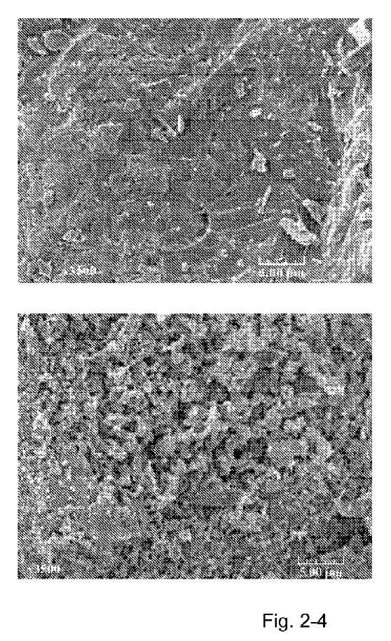

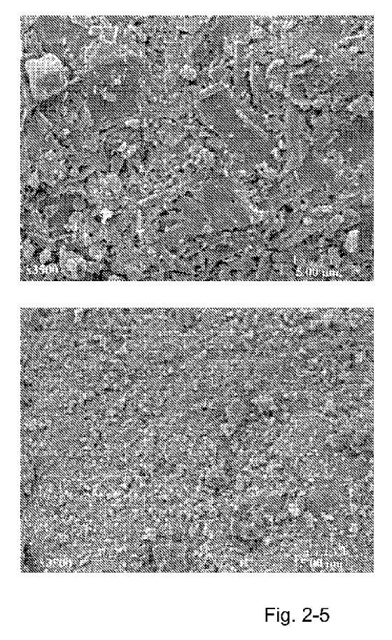

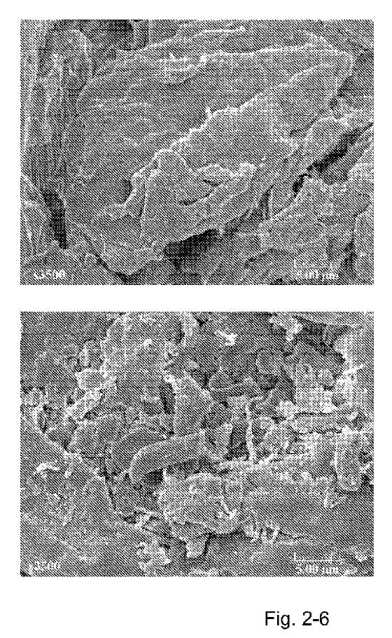

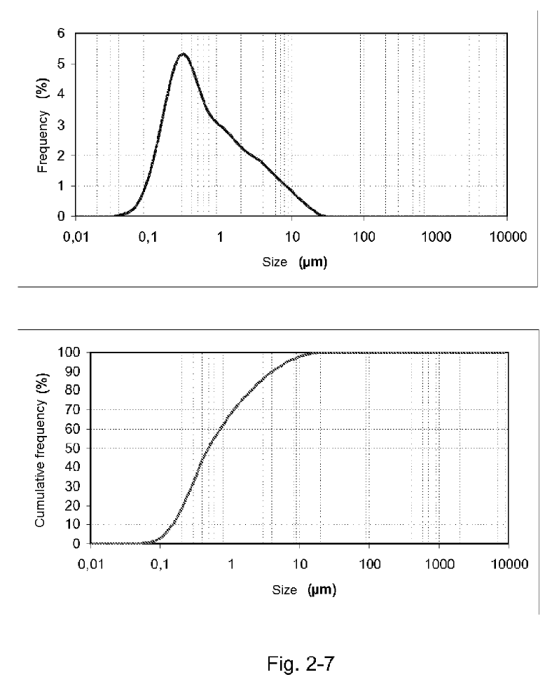

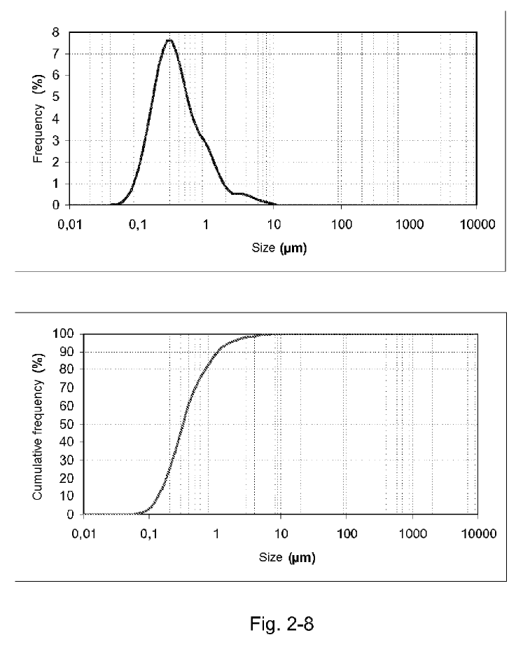

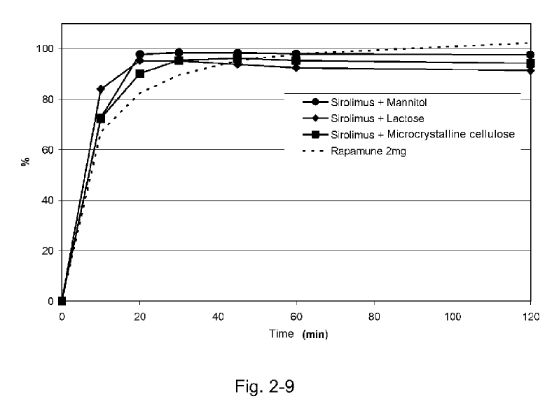

The results of differential scanning calorimetry (DSC) (FIG. 1-2) show that, for the two series of tests, a pressure of 3 MPa in the spraying enclosure produces a crystalline material in a solid state similar to that of the raw material, whereas spraying pressures of 1 MPa and 0.1 MPa produce sirolimus particles of lower degree of crystallinity. Indeed, the curves on FIG. 1-2 demonstrate an appreciable decrease in total enthalpy of the principal endothermic peak corresponding to the melting of sirolimus between 185° C. and 195° C. with the spraying pressure, regardless of the pre-expansion temperature. The sirolimus particles obtained with a spraying pressure of 1 MPa or 0.1 MPa are most probably comprised of the mixture of a crystallized phase and an amorphous phase.

Analysis by high-performance liquid chromatography indicates satisfactory stability of the sirolimus during the process for the tests conducted at a pre-expansion temperature of 60° C.: the titer and profile of the relative substances are similar to those of the starting compound for all of the samples. On the other hand, this same analysis reveals that the relative area of a chromatographic peak (relative retention time=1.56) for the tests carried out with a pre-expansion temperature of 140° C. is greater than for the starting compound. Moreover, it should be noted that this chromatographic peak is not observed for the sirolimus particles produced with a pre-expansion temperature of 60° C.

Consequently, it appears that particular conditions (pre-expansion temperature of 60° C. and pressure in the spraying enclosure of 3 MPa) make it possible to both preserve the crystallinity of the sirolimus and to ensure the stability of the compound compared to the starting compound during the process. On the other hand, these conditions lead to low collection yields and to powders that are difficult to handle.

### Example 2

### Production of Excipient/Compound (Sirolimus) Formulations by the RESS Method

The RESS method is implemented for the production of sirolimus particles deposited on an excipient placed beforehand in the spraying enclosure, with pilot-scale equipment corresponding to the diagram described in FIG. 1-1.

50 g of excipient in powder form (10) is first placed in the collection basket located in the spraying enclosure. 1.25 g of sirolimus (Batch S02) mixed with approximately 20 g of 1 mm-diameter glass beads is placed in a 20 ml extraction cell. Said cell is placed inside the extraction autoclave (5) heated at a temperature of 60° C. After initiation of the system, carbon dioxide in a supercritical state, at a pressure of 33 MPa, a temperature of 60° C. and a flow rate of 2.0 kg/h, percolates through the extraction cell to extract the sirolimus. The solution of sirolimus in supercritical carbon dioxide is released through a spray nozzle (8) comprised of a PEEK capillary tube with an internal diameter of 127 μm and a length suited to the pressure and flow rate of the system. The spraying enclosure (7), with a useful interior volume of 300 ml, is heated at 60° C. This same enclosure is equipped with a magnetic stirrer on whose shaft is adapted an anchor-type blade, which makes it possible, if need be, to uniformly distribute the excipient in the collection basket. The method is carried out at a pre-expansion temperature of 60° C. and a pressure in the spraying enclosure of 3 MPa, which are the optimized conditions from example 1 in terms of control of the solid state of the sirolimus and of the chemical stability of the compound during the process. After running for 5 hours, the CO₂ pump is stopped and the pressure in the spraying enclosure is gradually decreased to atmospheric pressure over approximately 30 minutes before the compound is collected.

Tests were carried out with three different excipients as indicated in table 2-1.

TABLE 2-1
Excipients used for tests 2-1 to 2-3

| | | Brand name of the |
|---|---|---|
| Test number | Type of excipient | excipient used |
| 2-1 | Mannitol | Pearlitol 200 SD |
| 2-2 | Lactose | Pharmatose DCL21 |
| 2-3 | Microcrystalline | Avicel PH 200 |
| | cellulose | |

Under the experimental conditions chosen, approximately 1.20 g of sirolimus was solubilized and sprayed on approximately 50 g of excipient during each test, which represents a theoretical load of sirolimus for each formulation of approximately 2.3% in mass. For the three excipients tested, the final formulation collected in the spraying enclosure basket is physically homogeneous (no agglomerates) and has the same appearance as the initial excipient particles.

Results

For each test, the sample collected was analyzed by:
differential scanning calorimetry (DSC) to characterize the solid state of the sirolimus within the formulation,
high-performance liquid chromatography (HPLC) to measure real loads and to verify the profile of the relative substances,
scanning electron microscopy (SEM) to observe the surface of the excipient particles as received and sirolimus/excipient formulations,
light-diffraction laser granulometry after dispersion of the formulations in an aqueous carrier and dissolution of the excipient to measure the size of the sirolimus particles in the formulation, only for formulations on mannitol and on lactose since cellulose cannot be solubilized in such an aqueous medium,
in vitro dissolution test compared to a reference formulation.

FIGS. 2-1 to 2-3 present a comparison of the thermograms of the pure excipient and the sirolimus/excipient formulation for the three tests. For each of the three sirolimus/excipient formulations, the thermogram of the formulation is very close to that of the excipient alone and does not demonstrate any particular anomaly. In each case, two endothermic peaks characteristic of sirolimus at approximately 183° C. and 195° C. can be identified and are indicative of the presence of crystalline sirolimus. A majority endothermic peak at 166° C. corresponding to that observed for the pure excipient appears in the case of mannitol.

The results of HPLC analysis demonstrate an elevated uniformity of load for the formulations (see table 2-2). In addition, the loads measured are very close to the theoretical load of 2.3% in mass, indicating that the implementation of the RESS method with capture on a bed of excipient makes it possible to collect sirolimus particles effectively. In addition, comparison of the profiles of the relative substances between sirolimus/excipient formulations obtained by the RESS method and physical, mixtures of starting sirolimus and corresponding excipient demonstrates the stability of the sirolimus deposited on the excipient by the method compared to the starting sirolimus.

TABLE 2-2
Results of load measurements for tests 2-1 to 2-3

| | Average | | Coefficient |
|---|---|---|---|
| Sample | Measured load | Standard | of variation |
| number | (%) | deviation | (%) |
| 2-1 | 2.07 | 0.04 | 1.9 |
| 2-2 | 2.00 | 0.05 | 2.3 |
| 2-3 | 2.41 | 0.01 | 0.4 |

FIGS. 2-4 to 2-6 present images obtained by scanning electron microscopy for samples 2-1 to 2-3, respectively. Except in the case of microcrystalline cellulose (FIG. 2-6), it appears very clearly that very fine sirolimus particles are deposited uniformly on the surface of the excipient particles. In the case of crystalline cellulose, the presence of very fine particles of sirolimus is difficult to observe and the relatively porous surface of the excipient particles suggests that the sirolimus particles are trapped inside crevices of the cellulose particles.

In the case of formulations deposited on mannitol and on lactose, the presence of very fine particles of sirolimus is confirmed by measurements taken with a light-diffraction laser granulometer in aqueous medium and after complete dissolution of the excipient particles. FIGS. 2-7 and 2-8 present the particle size distribution curves for the formulation on mannitol and the formulation on lactose, respectively. As indicated in table 2-3, the sirolimus particles obtained by the method of the invention are primarily sub-micron in size. In the case of the formulation on lactose, the implementation of the method is particularly effective in terms of production of particles of a volume mean diameter (dᵥ(0.5)) of a few hundred nanometers.

TABLE 2-3
Results of laser granulometry measurements
(average values of three tests)

| Test | Excipient | | | | |
|---|---|---|---|---|---|
| number | type | Dᵥ [4.3] | dᵥ (0.1) | dᵥ (0.5) | dᵥ (0.9) |
| 2-1 | Mannitol | 1.742 | 0.176 | 0.567 | 4.681 |
| 2-2 | Lactose | 0.647 | 0.159 | 0.371 | 1.277 |

In the context of the in vitro dissolution tests carried out on a USP I apparatus, the formulations were packaged into gelatin capsules (LGA, size 0, translucent, code 000020), in a quantity equivalent to 2 mg of sirolimus, and were analyzed by comparison with 2 mg Rapamune® tablets. The dissolution medium consisted of 0.4% SDS solution at a temperature of 37° C., with basket rotation set at 100 rpm. The samples were analyzed by HPLC/UV after filtration. The in vitro dissolution profiles presented in FIG. 2-9 show that the formulations in gelatin capsules have similar or better dissolution kinetics compared to the reference compound, Rapamune®.

The dissolution conditions presented in FIG. 2-9 are 0.4% sodium lauryl sulfate, 500 ml, basket at 100 rpm.

The sirolimus/excipient formulations obtained by the method of the invention, without the use of any solvent or surfactant and simply packaged in gelatin capsules, thus exhibit in vitro dissolution characteristics comparable to those of the reference formulation.

### Example 3

### Preparation and Characterization of Tablets from the Formulations of Example 2

Tablets were prepared from the excipient/compound formulation of example 2 and dosed with approximately 2 mg of sirolimus.

These tablets were manufactured on a rotary tablet press (SVIAC PR12) and the compression parameters were set to obtain tablets with a mass of roughly 337 mg.

The results compiled in table 3.1 show the satisfactory reproducibility of the tests in terms of sirolimus solubilization and collection yields.

TABLE 3-1
Tablet collection yields and characteristics

| | | Batch number | | | | | |
|---|---|---|---|---|---|---|---|
| | | XCXC6106 | | XCXC6107 | | XCXC6108 | |
| | | Excipients | | | | | |
| | | Mannitol | | Lactose | | Microcrystalline | |
| | | (Pearlitol 200 | | (Pharmatose | | cellulose | |
| | | SD) | | DCL21) | | (Avicel PH200) | |
| | | % | mg/tab | % | mg/tab | % | mg/tab |
| Excipient | Test 2.1 | 28.67 | 96.62 | — | — | — | — |
| | Test 2.2 | — | — | 29.67 | 100.00 | — | — |
| | Test 2.3 | — | — | — | — | 24.73 | 83.33 |
| Diluent | Mannitol | 71.13 | 239.71 | — | — | — | — |
| | Lactose | — | — | 70.13 | 236.33 | — | — |
| | Micro- | — | — | — | — | 75.07 | 253 |
| | crystalline | | | | | | |
| | cellulose | | | | | | |
| Lubricant | Magnesium | 0.20 | 0.68 | 0.20 | 0.68 | 0.20 | 0.67 |
| | stearate | | | | | | |

| | | | | | | |
|---|---|---|---|---|---|---|
| Total | 100.00 | 337.00 | 100.00 | 337.00 | 100.00 | 337.00 |

| | | | | |
|---|---|---|---|---|
| Formulation | Theoretical | 20.0 | 20.0 | 20.0 |
| titer | titer (mg/g) | | | |
| | Real titer | 20.7 | 20.0 | 24.1 |
| | (mg/g) | (CV = 1.9%) | (CV = 2.3%) | (CV = 0.4%) |
| Physical | Average | 336.9 | 337.0 | 338.3 |
| characteristics | mass (mg) | (CV = 0.50%) | (CV = ND) | (CV = 0.27%) |
| of the tablets | (n = 10) | | | |
| | Thickness | 4.44 | 4.23 | 5.46 |
| | (mm) | | | |
| | Hardness | 188.0 | 131.0 | 173.8 |
| | (N) (n = 10) | (CV = 9.0%) | (CV = 6.6%) | (CV = 3.6%) |
| Tablet titer | Theoretical | | | |
| | titer | 2.00 | 2.00 | 2.00 |
| | (mg/g) | | | |
| | Real titer | 1.81 | 2.05 | 2.11 |
| | (mg/g) | (CV = 1.0%) | (CV = 2.8%) | (CV = 1.5%) |
| | (n = 6) | | | |

CV: coefficient of variation expressed in %

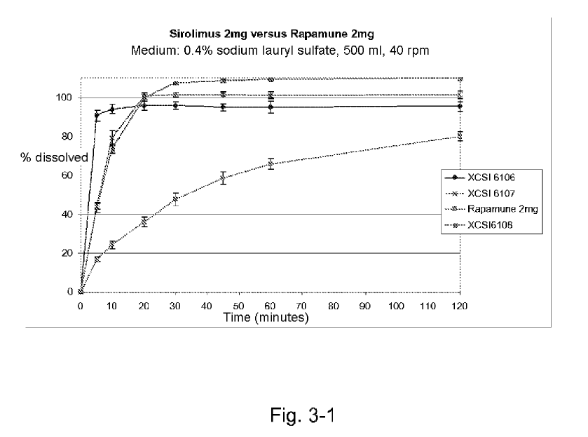

The dissolution conditions presented in FIG. 3-1 are 500 ml of medium comprised of 0.4% sodium lauryl sulfate, basket at 40 rpm.

The in vitro dissolution profiles (FIG. 3.1) show that the formulations in tablet form have better dissolution kinetics compared to the reference compound, Rapamune®.

1. A pharmaceutical formulation comprising particles of at least one pharmaceutically acceptable excipient on which are deposited fine crystalline particles of a “limus” family immunosuppressive macrolide.

2. The formulation according to claim 1, free of surface modifying agents and solvents.

3. The formulation according to claim 1, wherein the “limus” family immunosuppressive macrolide is selected from the group consisting of tacrolimus, sirolimus and analogues thereof.

4. The formulation according to claim 1, wherein the excipient is selected from the group consisting of a sugar, a polyol, starch, a cellulose derivative, magnesium aluminosilicate, and mixtures thereof.

5. The formulation according to claim 4, wherein the excipient is selected from the group consisting of lactose, microcrystalline cellulose, mannitol and mixtures thereof.

6. The formulation according to claim 4, wherein the load of the “limus” family immunosuppressive macrolide on pharmaceutically acceptable excipient particles is between 0.5% and 10%.

7. A pharmaceutical formulation according to claim 1 compounded for oral administration.

8. The pharmaceutical formulation according to claim 7, in gelatin capsule form or tablet form.

9. The pharmaceutical formulation according to claim 8, in gelatin capsule form.

10. The pharmaceutical formulation according to claim 8, in tablet form and wherein excipients are selected from the group consisting of diluents, binders, antistatic agents, lubricants, permeabilizing agents, pH modifiers, antioxidants, sweeteners, flavorings and mixtures thereof.

11. A method of effecting immunosuppressive treatment in a patient in need thereof comprising administering to said patient a pharmaceutical formulation comprising particles of at least one pharmaceutically acceptable excipient on which are deposited fine crystalline particles of a “limus” family immunosuppressive macrolide.

12. A method for preparing fine crystalline particles of “limus” family immunosuppressive macrolide comprising:
solubilizing a “limus” family immunosuppressive macrolide in CO₂, carried out at a pressure from 10 MPa to 100 MPa, and at a temperature from 35° C. to 80° C., in an extraction autoclave, and
depressurizing the solution obtained in the preceding step through a nozzle in a spraying enclosure, wherein the pressure in the spraying enclosure is from 1 MPa to 7 MPa.

13. A method for preparing a pharmaceutical formulation comprising:
solubilizing a “limus” family immunosuppressive macrolide in CO₂, at a pressure from 10 MPa to 40 MPa, and at a temperature from 35° C. and to 80° C., in an extraction autoclave, and
depressurizing the solution obtained in the preceding step through a nozzle in a spraying enclosure, wherein the pressure in the spraying enclosure is from 1 MPa and to 4 MPa, to produce fine crystalline particles of the “limus” family immunosuppressive macrolide; and
capturing the fine crystalline particles of the “limus” family immunosuppressive macrolide on pharmaceutically acceptable excipient.

14. The formulation according to claim 2, wherein the “limus” family immunosuppressive macrolide is selected from the group consisting of tacrolimus, sirolimus, and analogues thereof.

15. The formulation according to claim 2, wherein the excipient is selected from the group consisting of a sugar, a polyol, starch, a cellulose derivative, magnesium aluminosilicate, and mixtures thereof.

16. The formulation according to claim 3, wherein the excipient is selected from the group consisting of a sugar, a polyol, starch, a cellulose derivative, magnesium aluminosilicate, and mixtures thereof.

17. The formulation according to claim 6, wherein the load of the “limus” family immunosuppressive macrolide on pharmaceutically acceptable excipient particles is from 1% to 4%.

18. A pharmaceutical formulation of claim 2 compounded for oral administration.

19. A pharmaceutical formulation of claim 3 compounded for oral administration.

20. the method of claim 12, wherein: the solubilization of the “limus” family immunosuppressive macrolide in CO₂ is carried out at a pressure from 10 MPa to 40 MPa; and the depressurization of the solution is conducted at a pressure in the spraying enclosure from 1 MPa to 4 MPa.
